(19) United States
(12) Patent Application Publication
(10) Pub. No.: US 2004/0106687 A1
(43) Pub. Date: Jun. 3, 2004

(54) Carotenoids as anti-hypertension agents

(76) Inventors: Alon Harris, Beer Sheva (IL); Morris Zelkha, Beer Sheva (IL); Ester Paran, Beer Sheva (IL)

Correspondence Address: BROWDY AND NEIMARK, P.L.L.C., 624 NINTH STREET, NW, SUITE 300, WASHINGTON, DC, 20001-5303, US

(21) Appl. No.: 10/466,958

(22) PCT Filed: Jan. 21, 2002
(86) PCT No.: PCT/IL02/00054

(30) Foreign Application Priority Data
Jan. 23, 2001 (IL) 141038

(51) Int. Cl. A61K 31/015

(52) U.S. Cl. 514/763

(57) ABSTRACT

A composition for lowering blood pressure comprising an effective amount of a carotenoid selected from among a group consisting of lycopene, phytofluene, phytoene, astaxanthin and cathaxanthin or mixtures thereof. Further disclosed is a method for lowering blood pressure.

### FIELD OF THE INVENTION

The present invention relates to the field of carotenoid containing compositions and uses thereof in reducing blood pressure.

### BACKGROUND OF THE INVENTION

Carotenoids commonly occur in red, yellow, orange and green fruits and vegetables. Carotenoids, either of synthetic or natural sources, are known to be added to food and are taken as nutritional supplements. Lycopene, beta-carotene, phytofluene, phytoene, astaxanthin and cathaxanthin are among the carotenoids which have been found to demonstrate beneficial effects on human health, particularly on the vascular system.

U.S. Pat. No. 5,705,526 (Fujiwara, et al.) relates to a method for treating hypercholesterolemia in a patient in need thereof, which comprises administering to said patient a hypercholesterolemia therapeutic agent containing lycopene as an effective ingredient therein, wherein said lycopene is administered to said patient in an amount within a range of from 1 to 25 mg per day per adult. PCT/IL98/00286 in the name of Lycored Natural Industries Ltd. describes the use of a synergistic mixture of lycopene and vitamin E for inhibiting the oxidation of LDL in human blood, and thus effectively inhibiting the progression of atherosclerosis.

Hypertension, which effects a large number of the population, increases the risk of cardiovascular disease. Thus, there is much interest in methods for reducing blood pressure.

There are at least three known categories of methods for decreasing blood pressure in mammals:

1) Long term therapy on blood lipid profile—Treating atherosclerosis in order to increase the cross-sectional area of the blood vessel. Said treatment is usually via lowering LDL concentration and modifying LDL/HDL ratio in the blood, and preventing/inhibiting the oxidation of LDL in the blood.

2) Treatment of physical properties of the blood (e.g. lowering viscosity and inhibiting/preventing platelet aggregation);

3) Treatment of physical/mechanical properties of blood vessels (e.g. flexibility of the arterial wall and vasodilator response).

Hereinafter said categories referred to as Category 1, Category 2 and Category 3, respectively.

The effect of category 1 treatment on patients is usually noticeable after longer periods of treatment (e.g. more than 2 weeks), whereas treatment under categories 2 and 3 take effect within shorter periods of time (e.g. 2 days to 3 weeks).

Hypercholesterolemia and atherosclerosis which may cause hypertension, are treated according to category 1.

Galley et al, Clinical Science (1997) 92, 361-365, disclose an oral antioxidant synergistic combination comprising beta-carotene, vitamin E, vitamin C and other antioxidants, as effective in lowering blood pressure.

Therapeutic methods within Categories 2 and 3 employ drugs such as aspirin and vasodilating drugs such as nifedipine (hereinafter referred to as “conventional anti-hypertensive agents). There are undesirable side effects associated with the use of said drugs. There is therefore a need for a method for lowering blood pressure which is according to categories 2 and 3, which does have said side effects and is based on natural products.

Thus, it is an objective of the present invention to provide a method for lowering blood pressure by administering natural products, by a method which is not in category 1.

It is a further objective of the present invention to provide a composition for lowering blood pressure by a method which is not in category 1.

Other objectives of the present invention will become apparent as the description proceeds.

### SUMMARY OF THE INVENTION

According to one aspect, the present invention provides the use of a carotenoid selected from among a group consisting of lycopene, phytofluene, phytoene, astaxanthin and cathaxanthin, or mixtures thereof, for lowering blood pressure in a mammal, wherein said lowering of blood pressure is not according to Category 1.

According to a further aspect, the present invention provides a composition for lowering blood pressure in a mammal not according to Category 1, comprising a blood pressure lowering effective amount of a carotenoid selected from among a group consisting of lycopene, phytofluene, phytoene, astaxanthin and cathaxanthin or mixtures thereof.

According to yet a further aspect, the present invention relates to a method of lowering blood pressure in a mammal; said method not being a member of category 1, comprising administering to said mammal an effective amount of a carotenoid selected from a group consisting of lycopene, phytofluene, phytoene, astaxanthin and cathaxanthin, or mixtures thereof.

According to another aspect, the present invention relates to the use of a carotenoid selected from a group consisting of lycopene, phytofluene, phytoene, astaxanthin and cathaxanthin, or mixtures thereof, in the preparation of a medicament for lowering blood pressure in a human not according to Category 1.

According to another aspect, the present invention relates to a pharmaceutical composition useful in lowering blood pressure in a human not according to Category 1, comprising an effective amount of a carotenoid selected from a group consisting of lycopene, phytofluene, phytoene, astaxanthin and cathaxanthin, or mixtures thereof.

According to another aspect, the present invention relates to a solid dosage form useful in lowering blood pressure in a human by a method not being a member of Category 1, comprising an effective amount of a carotenoid selected from a group consisting of lycopene, phytofluene, phytoene, astaxanthin and cathaxanthin, or mixtures thereof.

In yet a further aspect of the present invention, a carotenoid selected from a group consisting of lycopene, phytofluene, phytoene, astaxanthin and cathaxanthin, or mixtures thereof, is added to functional foods, dietary supplements or drinks in order to lower blood pressure not according to Category 1.

### DETAILED DESCRIPTION OF PREFERRED EMBODIMENTS

The following description is illustrative of embodiments of the invention. The following description is not to be construed as limiting, it being understood that the skilled person may carry out many obvious variations to the invention.

It has unexpectedly been found that the administration of certain carotenoids effect a relatively quick response in the reduction of blood pressure (2 days to 2 weeks).

Throughout the specification the term carotenoids refers to carotenoids of natural, artificial and synthetic sources.

The following embodiments of the invention refer to the lowering of blood pressure not according to Category 1.

According to a particular embodiment of the method of the present invention, about 0.1 to 50 mg per day of a carotenoid selected from a group consisting of lycopene, phytofluene, phytoene, astaxanthin and cathaxanthin, or mixtures thereof, are administered to a subject in need of lowering blood pressure. The administration is preferably oral administration, however, all forms of administration which achieve a blood pressure lowering effective concentration of carotenoid in the blood, e.g. about 0.3 to 0.8 μM of lycopene, is suitable for the purposes of this invention. Administration may be by a single daily dose or multiple doses.

According to yet a further embodiment of the method of the present invention about 1-50 mg per day of lycopene are administered to a subject in need thereof. According to a particular aspect of the invention 5 to 20 mg are administered daily to a subject.

According to yet a preferred embodiment of the method of the present invention about 1 to 25 mg of lycopene are administered orally 2 times daily.

In yet a further embodiment of the method of the present invention about 3 to 50 mg of a mixture of carotenoids comprising lycopene (3%-15%), phytoene (0.3%-1%), phytofluene (0.3%-1%) is administered in order to lower blood pressure in a subject in need thereof. The foregoing mixture may further comprise beta-carotene (0.1%-0.3%) and vitamin E (0.5%-3%).

The composition according to the present invention comprises about 0.1-50 mg of a carotenoid selected from a group consisting of lycopene, phytofluene, phytoene, astaxanthin and cathaxanthin, or mixtures thereof. The composition may further comprise pharmaceutically acceptable adjuvant, excepients and additives. The composition may be in the form of tablets, capsules, hard shell capsules, gel-caps, soft gels or any further form suitable for the method of administration.

According to a preferred embodiment of the composition, said composition comprises 1 to 5 mg of lycopene.

In yet a further embodiment the composition of the present invention, the composition comprises about 3 to 50 mg of a mixture of carotenoids comprising lycopene (3%-15%), phytoene (0.3%-1%), phytofluene (0.3%-1%). The foregoing mixture may further comprise beta-carotene (0.1%-0.3%) and vitamin E (0.5%-3%).

In yet a further embodiment of the method of the present invention, the aforementioned dosages of carotenoids may be administered in conjunction with other conventional anti-hypertension agents. Accordingly, a pharmaceutical composition containing a conventional anti-hypertension agent and a carotenoid selected from a group consisting of lycopene, phytofluene, phytoene, astaxanthin and cathaxanthin, or mixtures thereof, is a further embodiment of the present invention.

According to yet a further embodiment of the present invention, a carotenoid selected from a group consisting of lycopene, phytofluene, phytoene, astakanthin and cathaxanthin, or mixtures thereof, may be added to food stuff, functional foods, dietary supplements and drinks in order to lower blood.

### EXAMPLES

### Example 1

Design and Methods: Thirty-five grade I HT subjects, aged 40-65, without concomitant diseases, who required no blood pressure and/or lipid lowering drug therapy, were recruited from primary care clinics. Study participants entered a two weeks run in period for establishment of HT (hypertension) diagnosis and base line evaluation, then 4 weeks placebo and finally 8 weeks treatment periods.

Results: Our results demonstrate significant reduction in both systolic BP (blood pressure) from 144 to 135, average of 9 mmHg reduction, and diastolic BP, from 91 to 84, average of 7 mmHg reduction.

### Example 2

A double-blind, placebo-controlled crossover study of lycopene consumption in 16 healthy young individuals. Each subject was studied at baseline and then given 15 mg lycopene or placebo for two weeks and re-evaluated. A one-month lycopene administration free washout period followed, another lycopene administration was administered prior to a final evaluation. Each evaluation included measures among other parameters also heart rate and blood pressure. During lycopene administration subjects showed a significant reduction in diastolic blood pressure as compared to placebo administration (p<0.05). Table 1 summarizes the results of this study.

TABLE 1

| Physiological parameter | Baseline | Placebo | Lycopene |
|---|---|---|---|
| Systolic Blood Pressure (mmHg) | 112 ± 3 | 111 ± 2 | 114 ± 2 |
| Diastolic Blood Pressure (mmHg) | 70 ± 2 | 70 ± 2 | 66 ± 2 |

While embodiments of the invention have been described by way of illustration, it will be apparent that the invention may be carried out with many modifications, variations and adaptations, without departing from its spirit or exceeding the scope of the claims.

1) A method for lowering blood pressure in a mammal, said method not being a member of Category 1, comprising administering to said mammal a blood pressure lowering effective amount of a carotenoid selected from a group consisting of lycopene, phytofluene, phytoene, astaxanthin and cathaxanthin, or mixtures thereof.

2) A method according to claim 1 in which said mammal is human.

3) A method according to claim 1 wherein the carotenoid is lycopene.

4) A method according to claim 1 wherein about 0.1-50 mg of a carotenoid or mixture of carotenoids are administered.

5) A method according to claim 4 wherein about 5-20 mg of a carotenoid or mixture of carotenoids are administered daily.

6) A method according to claim 1 wherein about 1-25 mg of lycopene are administered daily.

7) A method according to claim 1 wherein a carotenoid or mixture of carotenoids is administered in conjunction with a conventional anti-hypertensive agent.

8) A method according to claim 7 wherein lycopene is administered in conjunction with a conventional anti-hypertensive agent.

9) A method according to claim 1 wherein a mixture of carotenoids is administered.

10) A method according to claim 9 wherein the mixture of carotenoids comprises lycopene, phytoene and phytofluene.

11) A method according to claim 10 wherein the carotenoid mixture comprises about 3%-15% lycopene, 0.3%-1% phytoene and 0.3%-1% phytofluene.

12) A method according to claim 11 wherein about 3 to 30 mg of mixture are administered daily.

13) Use of a carotenoid selected from among a group consisting of lycopene, phytofluene, phytoene, astaxanthin and cathaxanthin, in the preparation of a composition for lowering blood pressure in a mammal, wherein said lowering of blood pressure is not according to Category 1.

14) Use according to claim 13 in which said mammal is human.

15) Use according to claim 13 wherein the carotenoid is lycopene.

16) Use according to claim 13 wherein 0.1-50 mg of a carotenoid or mixture of carotenoids is applied.

17) Use according to claim 13 wherein about 1-25 mg of lycopene is applied.

18) Use according to claim 13 wherein a carotenoid or mixtures thereof are applied with a conventional anti-hypertensive agent.

19) Use according to claim 13 wherein lycopene is applied with a conventional anti-hypertensive agent.

20) A composition for lowering blood pressure in a human by a method not being a member of Category 1, comprising an effective amount of a carotenoid selected from among a group consisting of lycopene, phytofluene, phytoene, astaxanthin and cathaxanthin, or mixtures thereof.

21) A composition according to claim 20 comprising an effective amount of lycopene.

22) A composition according to claim 20 comprising about 0.1 to 50 mg of a carotenoid or a mixture of carotenoids and a pharmaceutically acceptable adjuvant, excepient or additive.

23) A composition according to claim 20 comprising a mixture of carotenoids.

24) A composition according to claim 23 wherein the mixture of carotenoids comprises lycopene, phytoene and phytofluene.

25) A composition according to claim 24 comprising about 3%-15% lycopene, 0.3%-1% phytoene and 0.3%-1% phytofluene.

26) A composition according to claim 25 comprising about 3 to 30 mg of carotenoid mixture.

27) A composition according to claim 20, wherein said composition is intended for oral administration.

28) A composition according to claim 20 in the form of tablets, capsules, hard shell capsules, gel caps or soft gels.

29) A composition according to claim 20 further comprising a conventional anti-hypertensive agent.

30) A solid dosage form comprising an effective amount of lycopene useful in lowering blood pressure in a human by a method not being a member of category 1.

31) Use of a composition as described in claim 20 as an additive to food stuff, functional foods, dietary supplements and drinks.

32) A pharmaceutical composition for lowering blood pressure in a mammal, not according to Category 1, comprising an effective amount of a carotenoid selected from a group consisting of lycopene, phytofluene, phytoene, astaxanthin and cathaxanthin, or mixtures thereof.

33) A pharmaceutical composition according to claim 32 wherein the carotenoid is lycopene.

34) A pharmaceutical composition according to claim 32 comprising about 0.1-50 mg of a carotenoid or mixture of carotenoids.

35) A pharmaceutical composition according to claim 32 further comprising a conventional anti-hypertensive agent.

36) A pharmaceutical composition according to claim 32 comprising a mixture of carotenoids.

37) A pharmaceutical composition according to claim 36 wherein the mixture of carotenoids comprises lycopene, phytoene and phytofluene.

38) A pharmaceutical composition according to claim 37 wherein the carotenoid mixture comprises about 3%-15% lycopene, 0.3%-1% phytoene and 0.3%-1% phytofluene.

39) A composition according to claim 36 comprising about 3 to 30 mg of mixture of carotenoids.

40) A method substantially as described and exemplified.

41) A use substantially as described and exemplified.

42) A composition substantially as described and exemplified.
